(19) United States
(12) Patent Application Publication
(10) Pub. No.: US 2015/0272508 A1
(43) Pub. Date: Oct. 1, 2015

(54) SIGNAL PROCESSING SYSTEM PROVIDING MARKING OF LIVING CREATURE PHYSIOLOGICAL SIGNAL AT A SPECIFIC TIME

(71) Applicant: UE Technology, Taichung (TW)

(72) Inventors: MIN-HUI CHIOUCHANG, Taichung (TW); HAO-YI HUNG, Taichung (TW); SHENG-CHUAN LIANG, Taichung (TW); YUNG-JIUN LIN, Taichung (TW)

(73) Assignee: UE Technology, Taichung (TW)

(21) Appl. No.: 14/227,133

(22) Filed: Mar. 27, 2014

(51) Int. Cl. A61B 5/00; A61B 5/0402; A61B 5/145; A61B 5/0488; A61B 5/11; A61B 5/021; A61B 5/0476

(52) U.S. Cl. CPC A61B 5/7246; A61B 5/021; A61B 5/0402; A61B 5/0476; A61B 5/0488; A61B 5/11; A61B 5/14542; A61B 2560/0242

(57) ABSTRACT

A signal processing system aims to mark living creature physiological signal at a specific time by monitoring living creature's explicit characteristic behavior or ambient environment variation. It includes a trigger device to trigger a time marking device to generate a time signal, a signal processing device to receive the time signal through a transmission interface, and a detection device to measure and send a physiological signal of the living creature to the signal processing device. The signal processing device compensates delay time of the transmission interface, trigger device and time marking device to get a corrected time signal. Thus, the signal processing device can add the corrected time signal in the physiological signal or capture the physiological signal corresponding to a specific time of the corrected time signal to correctly interpret correlation between the living creature's explicit characteristic behavior or ambient environment variation and the physiological signal.

### FIELD OF THE INVENTION

The present invention relates to measurement of living creature physiological signal and particularly to a signal processing system capable of marking a living creature's physiological signal at a specific time through monitoring living creature's explicit characteristic behavior or ambient environment variation.

### BACKGROUND OF THE INVENTION

When a living creature generates a stimulation signal when encountering ambient environment variation, such as sound, light, heat or the like, a series of physiological signal alterations often ensue, such as change of brainwave signals, electrocardiographic signals, electromyography signals, and the like. Meanwhile, the living creature could generate corresponding explicit characteristic behavior. The stimulation signal, depending on its nature, can stimulate the living creature in different ways. Take a human body for instance, if the stimulation signal is a transient blaze, the blaze stimulates the retina of the human body and the optic nerve on the retina generates neural signal alteration sent to the visual cortex of the brain; upon a judgment is made, another neural signal is generated to control the eyelid closing muscle group, thereby an explicit action of eyelid closing is generated to avoid the eyes from being injured by the blaze. By measuring or recording the stimulation signal and the brainwave signal, eyelid electromyography signal and explicit characteristic behavior that are caused by the stimulation signal, the impact of the stimulation to the living creature can be understood.

There are also patients who suffer from special illnesses, such as epilepsy, Parkinson's disease or the like. When the patients show notable explicit characteristic behavior, symptoms of the illness can be easily recognized and necessary treatments can be then taken. In the conventional approach, in order to get correlation between the physiological signals when the illness breaks out and the explicit characteristic behavior, the patient's physiological signals have to be monitored and measured for a long time. When the notable explicit characteristic behavior is shown, the obtained physiological signals are analyzed to interpret the correlation between the physiological signals and explicit characteristic behavior to find out timely the illness through change of the physiological signals so that proper actions can be taken instantly to maintain the health of the patient.

However, the conventional technique of interpreting the correlation between the physiological signals and explicit characteristic behavior cannot accurately get the corresponding correlation time between them, namely when the explicit characteristic behavior takes place, only the rough time corresponding to the physiological signals occur can be known. This causes problem in interpreting the correlation between the physiological signals and explicit characteristic behavior, and could also result in faulty judgment of the correlation between them because of the incorrect correlation time to affect following analysis and interpretation.

### SUMMARY OF THE INVENTION

Therefore the primary object of the present invention is to provide a signal processing system to mark a living creature's physiological signal at a specific time through a trigger device which monitors living creature's explicit characteristic behavior or ambient environment variation so that correct correspondence between the captured physiological signal and the living creature's explicit characteristic behavior or ambient environment variation can be made to correctly interpret the correlation between the living creature's explicit characteristic behavior or ambient environment variation and the physiological signal.

To achieve the foregoing object, the signal processing system capable of marking living creature physiological signal at a specific time is provided to measure a physiological signal of a living creature. It comprises a trigger device, a time marking device, a detection device, a signal processing device and a transmission interface. The trigger device generates an operation signal. The time marking device is connected to the trigger device to receive the operation signal and generate a time signal. The detection device measures a physiological signal of the living creature. The signal processing device is connected to the detection device to receive the physiological signal. The transmission interface is connected to the signal processing device and time marking device so that the signal processing device can receive the time signal. The signal processing device compensates delay time of the transmission interface, trigger device and time marking device through signal processing to get a corrected time signal and obtain a correct time at which the physiological signal occurs according to the corrected time signal. Moreover, the signal processing device adds the corrected time signal in the captured physiological signal, or captures the physiological signal corresponding to a specific time of the corrected time signal.

Thus, whenever the living creature generates explicit characteristic behavior or ambient environment variation occurs, the trigger device generates the operation signal and the time marking device generates the time signal sent to the signal processing device so that the signal processing device can get the corrected time signal and add the corrected time signal in the physiological signal, or capture the physiological signal corresponding to a specific time of the corrected time signal, thereby the physiological signal can correctly correspond to the living creature's explicit characteristic behavior and ambient environment variation, and correct interpretation of the correlation between the living creature's explicit characteristic behavior or ambient environment variation and the physiological signal can be made to meet use requirement.

In the invention, it is not limited to measure only one physiological signal of one living creature, but also can measure multiple physiological signals from different regions of one living creature. In this embodiment, a plurality of detection devices and a plurality of signal processing devices are provided. Each detection device is used to measure one physiological signal from one region of the living creature, and each signal processing device is connected to the detection device to receive the physiological signal and process the captured physiological signal according to the corrected time signal. Alternatively, the invention also can use the detection devices to measure multiple physiological signals of living creatures or measure multiple physiological signals from different regions of living creatures, and use the signal processing devices to process the physiological signals.

At certain time, it may be necessary to monitor multiple living creatures at the same time for observing their social interaction and/or coherent behavior in response to the same exogenous stimulation. For example (not limited to), we may want to measure the brain responses using brain wave (EEG) devices on multiple human subjects while they are watching the same video clip or listening to the same music excerpt. In such a case, several signal processing devices may be used to separately collect EEG signal from multiple human subjects at the same time. In order to make precise timing information to synchronize the EEG recordings from all the participants, the single time marking device can create the trigger signals and send to all signal processing devices simultaneously to synchronize the time markers across all recordings.

The foregoing, as well as additional objects, features and advantages of the invention will be more readily apparent from the following detailed description, which proceeds with reference to the accompanying embodiments and drawings.

The embodiments serve merely for illustrative purpose and are not the limitation of the invention.

### BRIEF DESCRIPTION OF THE DRAWINGS

FIG. 1 is a block diagram of the signal processing system of the invention.

FIG. 2 is a block diagram of a first embodiment of the invention.

FIG. 3 is a block diagram of a second embodiment of the invention.

FIG. 4 is a block diagram of a third embodiment of the invention.

FIG. 5 is a block diagram of a fourth embodiment of the invention.

FIG. 6 is a block diagram of a fifth embodiment of the invention.

FIG. 7 is a block diagram of a sixth embodiment of the invention.

FIG. 8 is a block diagram of a seventh embodiment of the invention.

### DETAILED DESCRIPTION OF THE PREFERRED EMBODIMENTS

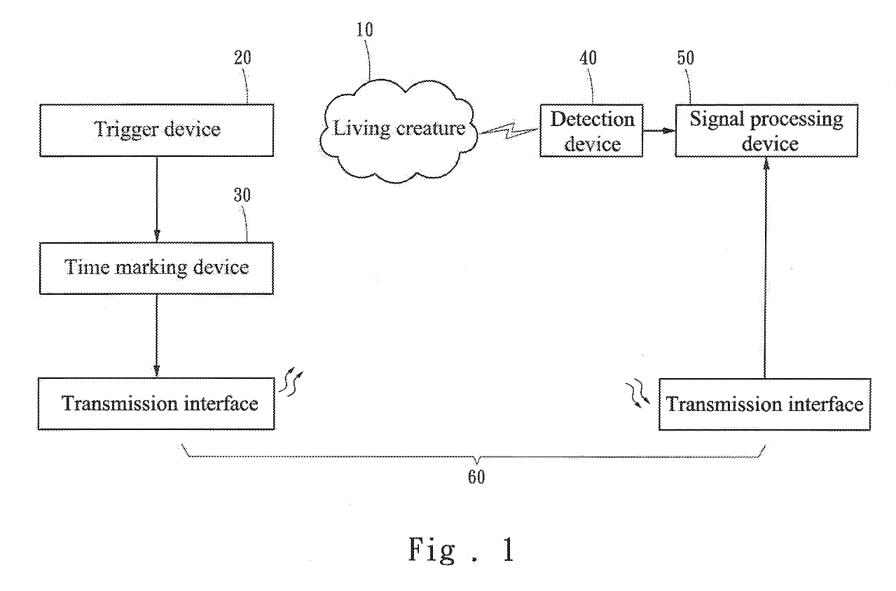

Please refer to FIG. 1, the present invention aims to provide a signal processing system capable of marking living creature physiological signal at a specific time that is used to measure a physiological signal of a living creature 10. It comprises a trigger device 20, a time marking device 30, a detection device 40, a signal processing device 50 and a transmission interface 60. The trigger device 20 generates an operation signal. The time marking device 30 is connected to the trigger device 20 to receive the operation signal and generate a time signal. The detection device 40 measures a physiological signal of the living creature 10. The signal processing device 50 is connected to the detection device 40 to receive the physiological signal. The transmission interface 60 is connected to the signal processing device 50 and time marking device 30 so that the signal processing device 50 can receive the time signal. The signal processing device 50 compensates delay time of the transmission interface 60, trigger device 20 and time marking device 30 through signal processing to get a corrected time signal and obtain a correct time at which the physiological signal occurs according to the corrected time signal. The signal processing device 50 also adds the corrected time signal in the captured physiological signal or captures the physiological signal corresponding to a specific time of the corrected time signal.

In practice, the trigger device 20 can be implemented in many styles. The following includes embodiments 1 through 4 as examples for discussion.

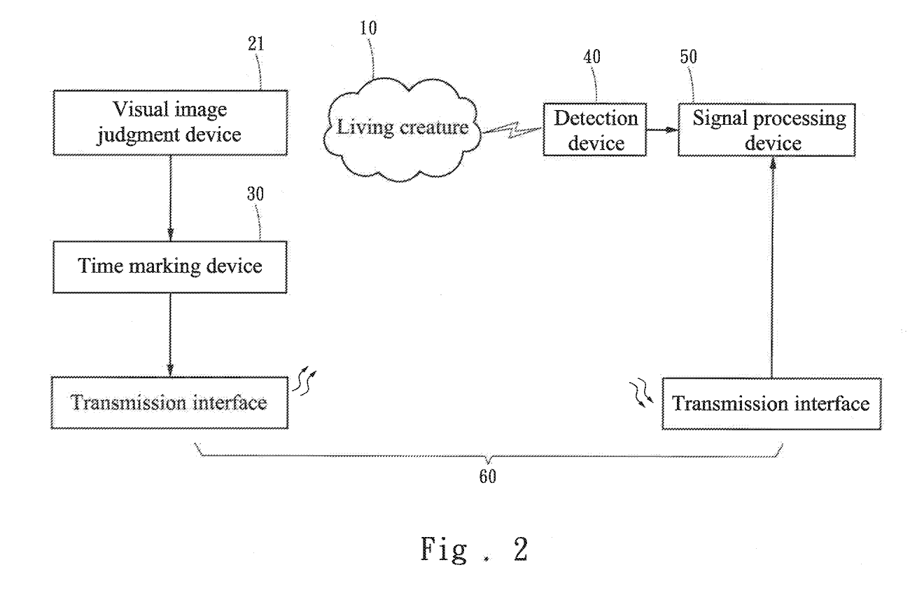

Please refer to FIG. 2 for a first embodiment in which the trigger device 20 is a visual image judgment device 21 to monitor motion images of the living creature 10 or ambient environment images. When the visual image judgment device 21 judges that an explicit characteristic behavior of the living creature 10 or ambient environment variation has occurred, it generates the operation signal.

More specifically, the visual image judgment device 21 can consist of a video camera and an image processing unit. The video camera captures images of the living creature 10 and the ambience. The image processing unit judges whether change occurs to the images of the living creature 10 and ambient environment. For instance, in the event that the living creature 10 is trembling, the image processing unit can directly judge that an explicit characteristic behavior of the living creature 10 has taken place. It is to be noted that the judgment principle of the image processing unit is set in advance. Users can set different image variations according to requirements to judge whether the explicit characteristic behavior of the living creature 10 or ambient environment variation occurs.

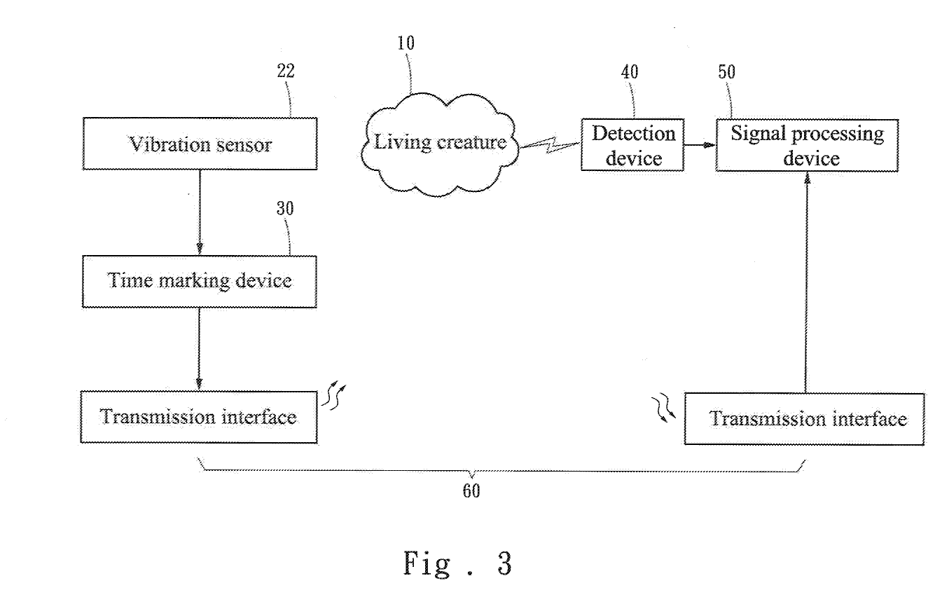

Please refer to FIG. 3 for a second embodiment in which the trigger device 20 is a vibration sensor 22 carried by the living creature 10. When the vibration sensor 22 detects that the living creature 10 is trembling, the living creature 10 is deemed to have an explicit characteristic behavior, and then the vibration sensor 22 generates the operation signal.

The vibration sensor 22 can be a gyroscope, accelerometer, magnetometer, inclination sensor, pressure sensor, optical sensor, image identification device, sound identification device or the like, and aims to detect whether an explicit characteristic behavior such as vibration of the living creature 10 occurs. Hence when the vibration sensor 22 worn by the living creature 10 detects an action thereof that conforms to a preset condition, a judgment of an abrupt event can be made, such as trembling, falling down, abrupt stopped movement or the like. Thereby an explicit characteristic behavior of the living creature 10 can be deemed to take place. The preset condition can be set according to the status of the living creature 10. In addition, since a general smartphone is equipped with a gyroscope, accelerometer or the like, the vibration sensor 22 can be directly implemented via the smartphone.

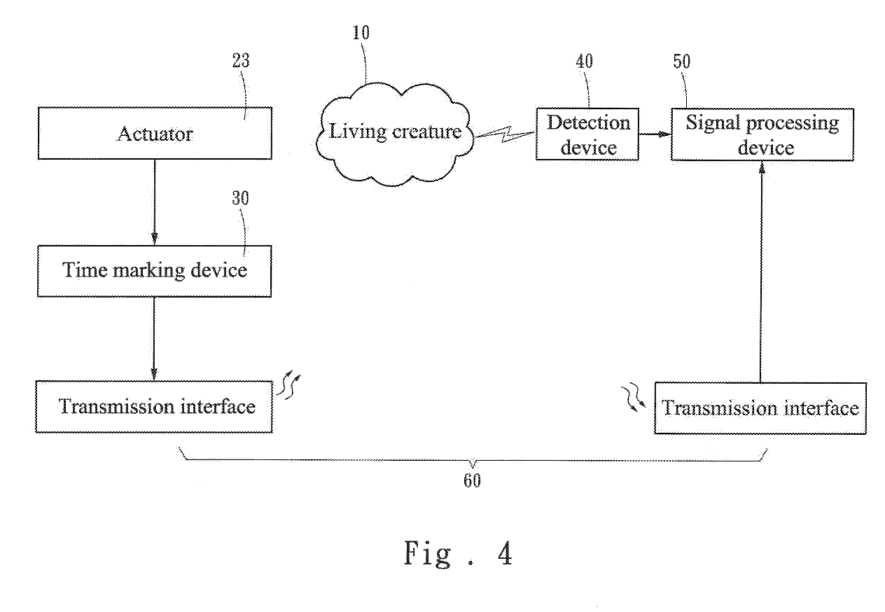

Please refer to FIG. 4 for a third embodiment in which the trigger device 20 is an actuator 23 controlled by a holder. In the event that an explicit characteristic behavior of the living creature 10 or ambient environment variation is observed by the holder of the actuator 23, the holder can trigger the actuator 23 to generate the operation signal. The actuator 23 can be a mechanical switch, a touch-control switch, a touch-slide switch, a proximity switch, a capacitor switch, an optical induction switch, a sound induction switch or the like.

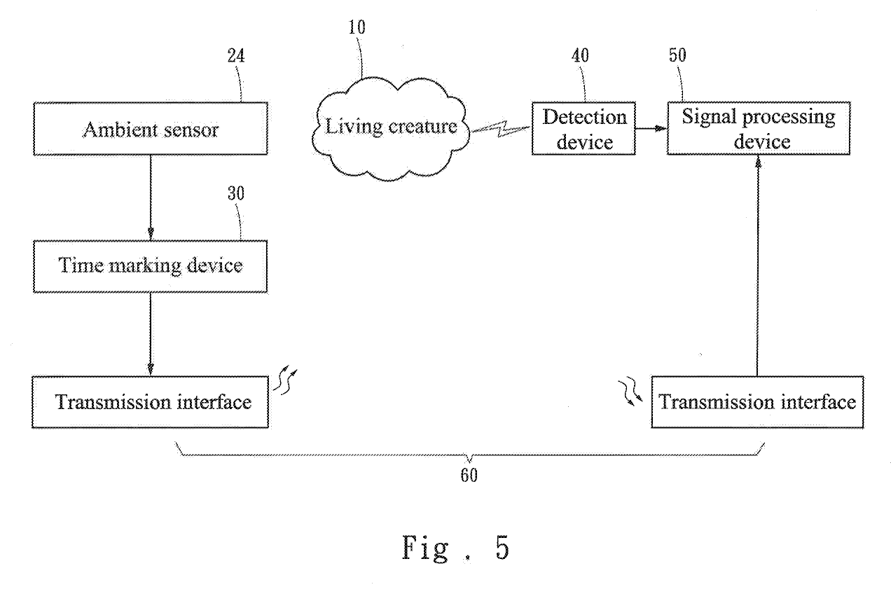

Please refer to FIG. 5 for a fourth embodiment in which the trigger device 20 is an ambience sensor 24 which generates the operation signal upon detecting that ambient environment variation conform to a preset condition. The ambience sensor 24 can detect parameters selected from the group consisting of light, radiation, sound, magnetic field, electric field, flow amount, temperature, humidity, gas and pressure. For instance, the ambience sensor 24 can be set to generate the operation signal when the temperature is higher than a preset value, or the humidity is higher than a preset value, or a specific gas is detected, or the atmospheric pressure is in a specific range. Moreover, the preset condition for the ambience sensor 24 to generate the operation signal can be set according to the status of the living creature 10 to meet actual requirement. Namely, when the explicit characteristic behavior of the living creature 10 is generated due to the ambient environment variation, the ambience sensor 24 can generate the operation signal.

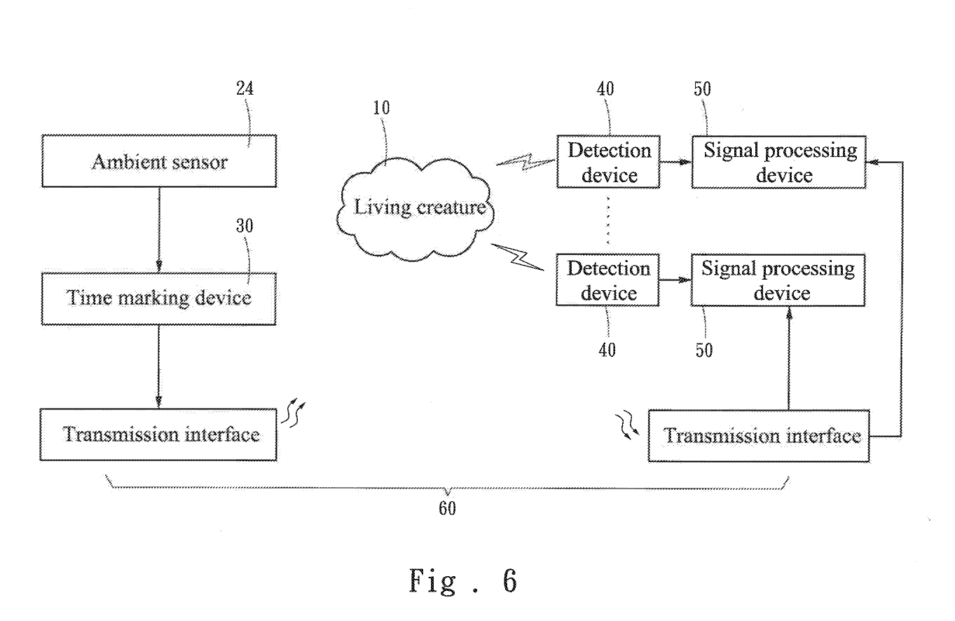

Please refer to FIG. 6 for a fifth embodiment to measure multiple physiological signals from different regions of a living creature 10. In this embodiment, it differs from the previous embodiments by providing a plurality of detection devices 40 and a plurality of signal processing devices 50. Each detection device 40 is to measure one physiological signal from one region of the living creature 10. Each signal processing device 50 is connected to the detection device 40 to receive the physiological signal. The transmission interface 60 is connected to the multiple signal processing devices 50 and the time marking device 30. The signal processing device 50 also receives the time signal time marking device 3 and then compensates the delay time of the transmission interface 60, the trigger device 20 and the time marking device 30 to get a corrected time signal and obtain a correct time at which the physiological signal occurs according to the corrected time signal to allow the signal processing device 50 to process the captured physiological signal.

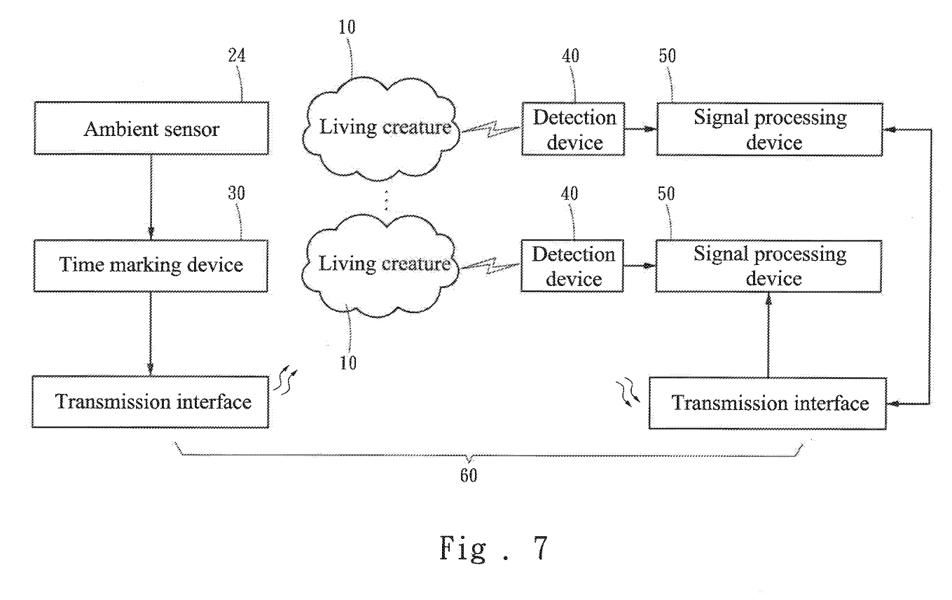

Please refer to FIG. 7 for a sixth embodiment to measure multiple physiological signals of a plurality of living creatures 10. In this embodiment, it also provides a plurality of detection devices 40 and a plurality of signal processing devices 50. Each detection device 40 measures one physiological signal of one living creature 10. Each signal processing device 50 is connected to the detection device 40 to receive the physiological signal. The transmission interface 60 is connected to the multiple signal processing devices 50 and the time marking device 30. The signal processing device 50 also receives the time signal from the time marking device 3 and then compensates the delay time of the transmission interface 60, the trigger device 20 and the time marking device 30 to get a corrected time signal and obtain a correct time at which the physiological signal occurs according to the corrected time signal to allow the signal processing device 50 to process the captured physiological signal.

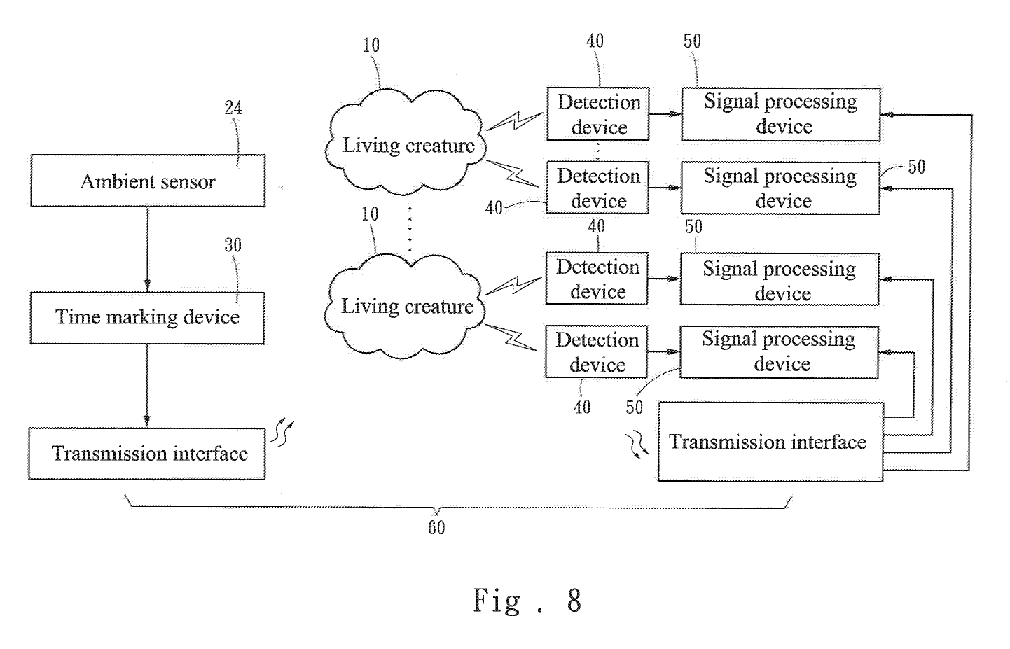

Please refer to FIG. 8 for a seventh embodiment to measure multiple physiological signals from different regions of a plurality of living creatures 10. In this embodiment, it also provides a plurality of detection devices 40 and a plurality of signal processing devices 50. Each detection device 40 measures one physiological signal from one region of one living creature 10. Each signal processing device 50 is connected to the detection device 40 to receive the physiological signal. The transmission interface 60 is connected to the multiple signal processing devices 50 and the time marking device 30. The signal processing device 50 also receives the time signal from the time marking device 3 and then compensates the delay time of the transmission interface 60, the trigger device 20 and the time marking device 30 to get a corrected time signal and obtain a correct time at which the physiological signal occurs according to the corrected time signal to allow the signal processing device 50 to process the captured physiological signal.

The transmission interface 60 can be a wireless transmission interface selected from the group consisting of WiFi, WiMAX, LTE, UWB, ZigBee, Bluetooth, microwave, infrared ray and radio frequency. Other wireless transmission techniques not mentioned above also are adoptable and shall be included in the scope of the invention to improve usability. The connection link can also be made in a wired connection fashion.

The detection device 40 of the invention can be selected from the group consisting of a motion sensor, a brainwave sensor, an electromyography sensor, an electrocardiography sensor, a blood pressure sensor, a blood oxygen sensor, an impedance sensor and a body thermometer, and can be chosen according to requirement of the living creature 10.

As a conclusion, the invention can generate individual or multiple operation signals through various trigger devices when explicit characteristic behavior of the living creature or ambient environment variation takes place, or in incorporation with a system which can detect multiple explicit characteristic behaviors and multiple ambient environment variations. The time marking device can generate a time signal corresponding to each operation signal to allow the signal processing device to get the corrected time signal, and then add the corrected time signal to the physiological signal or capture a physiological signal corresponding to a specific time of the corrected time signal. Hence the physiological signal can correctly correspond to the living creature's elicit characteristic behavior, and consequentially can make correct interpretation of the correlation between the living creature's explicit characteristic behavior or ambient environment variation and the physiological signal to meet use requirement.

What is claimed is:

1. A signal processing system providing marking of living creature physiological signal at a specific time to measure a physiological signal of a living creature, comprising:
a trigger device for generating an operation signal;
a time marking device connected to the trigger device to receive the operation signal and generate a time signal;
a detection device for measuring the physiological signal of the living creature;
a signal processing device connected to the detection device to receive the physiological signal; and
a transmission interface connected to the signal processing device and the time marking device;
wherein the signal processing device receives the time signal and compensates delay time of the transmission interface, the trigger device and the time marking device through signal processing to get a corrected time signal and obtain a correct time at which the physiological signal occurs according to the corrected time signal to allow the signal processing device to process the captured physiological signal.

2. The signal processing system of claim 1, wherein the signal processing device adds the corrected time signal in the captured physiological signal.

3. The signal processing system of claim 1, wherein the physiological signal captured by the signal processing device corresponds to a specific time of the corrected time signal.

4. The signal processing system of claim 1, wherein the trigger device is a visual image judgment device to monitor motion images of the living creature and generate the operation signal upon judging that an explicit characteristic behavior of the living creature has occurred.

5. The signal processing system of claim 1, wherein the trigger device is a visual image judgment device to monitor ambient environment images of the living creature and generate the operation signal upon judging that variation of the ambient environment images has occurred.

6. The signal processing system of claim 1, wherein the trigger device is a vibration sensor wearable by the living creature to generate the operation signal upon judging that an explicit characteristic behavior of the living creature has occurred.

7. The signal processing system of claim 1, wherein the trigger device is an actuator controllable by a holder and triggered by the holder to generate the operation signal when an explicit characteristic behavior of the living creature has been observed by the holder.

8. The signal processing system of claim 1, wherein the trigger device is an actuator controllable by a holder and triggered by the holder to generate the operation signal when variation of an ambient environment of the living creature has been observed by the holder.

9. The signal processing system of claim 1, wherein the trigger device is an ambience sensor to generate the operation signal upon detecting that variation of an ambient environment conform to a preset condition.

10. A signal processing system providing marking of living creature physiological signal at a specific time to measure physiological signals from different regions of a living creature, comprising:
a trigger device for generating an operation signal;
a time marking device connected to the trigger device to receive the operation signal and generate a time signal;
a plurality of detection devices each measuring one physiological signal from one region of the living creature;
a plurality of signal processing devices each being connected to one detection device to receive the physiological signal; and
a transmission interface connected to the plurality of signal processing devices and the time marking device;
wherein each signal processing device receives the time signal and compensates delay time of the transmission interface, the trigger device and the time marking device through signal processing to get a corrected time signal and obtain a correct time at which the physiological signal occurs according to the corrected time signal to allow the signal processing device to process the captured physiological signal.

11. A signal processing system providing marking of living creature physiological signal at a specific time to measure physiological signals of living creatures, comprising:
a trigger device for generating an operation signal;
a time marking device connected to the trigger device to receive the operation signal and generate a time signal;
a plurality of detection devices each measuring one physiological signal of one living creature;
a plurality of signal processing devices each being connected to one detection device to receive the physiological signal; and
a transmission interface connected to the plurality of signal processing devices and the time marking device;
wherein each signal processing device receives the time signal and compensates delay time of the transmission interface, the trigger device and the time marking device through signal processing to get a corrected time signal and obtain a correct time at which the physiological signal occurs according to the corrected time signal to allow the signal processing device to process the captured physiological signal.

12. A signal processing system providing marking of living creature physiological signal at a specific time to measure physiological signals from different regions of living creatures, comprising:
a trigger device for generating an operation signal;
a time marking device connected to the trigger device to receive the operation signal and generate a time signal;
a plurality of detection devices each measuring one physiological signal from one region of one living creature;
a plurality of signal processing devices each being connected to one detection device to receive the physiological signal; and
a transmission interface connected to the plurality of signal processing devices and the time marking device;
wherein each signal processing device receives the time signal and compensates delay time of the transmission interface, the trigger device and the time marking device through signal processing to get a corrected time signal and obtain a correct time at which the physiological signal occurs according to the corrected time signal to allow the signal processing device to process the captured physiological signal.
